(19) United States
(12) Patent Application Publication
(10) Pub. No.: US 2010/0256735 A1
(43) Pub. Date: Oct. 7, 2010

(54) INTRALUMINAL STENT WITH SEAM

(75) Inventor: Rolando Morales, JR., League City, TX (US)

Correspondence Address: LOCKE LORD BISSELL & LIDDELL LLP, 600 TRAVIS SUITE 2800, HOUSTON, TX, 77002-3095, US

(73) Assignee: Board of Regents, The University of Texas System, Austin, TX (US)

(21) Appl. No.: 12/732,553

(22) Filed: Mar. 26, 2010

Related U.S. Application Data
(60) Provisional application No. 61/166,329, filed on Apr. 3, 2009.

(51) Int. Cl. A61F 2/82; A61F 2/88

(52) U.S. Cl. 623/1.15; 623/1.22

(57) ABSTRACT

A disclosure provides a removable intraluminal stent for treating injuries or other conditions inside a body lumen. The stent can include a seam, such as a helical seam, for supporting the structure of the stent, which can be unseamed, for example, during removal of the stent from a lumen. The stent can include an elongated tubular member, or stent body, for positioning inside a lumen. The stent can be flexible and can include one or more other stent components coupled to the body. The stent can be formed from an obliquely-angled-parallelogram-shaped sheet. The stent can include a peripheral wire, which can be memory shape-retaining wires, for coupling the stent in place and/or a longitudinal support wire for supporting one or more components of the stent.

### CROSS REFERENCE TO RELATED APPLICATIONS

This application claims the benefit of U.S. Provisional Patent Application No. 61/166,329, filed on Apr. 3, 2009.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

1. Field of the Invention

The invention disclosed and taught herein relates generally to stents; and more specifically relates to removable intraluminal stents.

2. Description of the Related Art

The use of stent medical devices, or other types of endoluminal mechanical support devices, to keep a duct, vessel or other body lumen open in a body has developed into a primary therapy for damaged or perforated endoluminal walls. The use of stents in various medical procedures has quickly become accepted as experience with stent devices accumulates, and the number of medical procedures employing them increases as their advantages become more widely recognized. For example, it is known to use stents in body lumens in order to maintain open passageways or protect damaged endoluminal walls, such as in the prostatic urethra, the esophagus, the biliary tract, intestines, various coronary arteries and veins or for any endluminal anastomosis, as well as more remote cardiovascular vessels such as the femoral artery, etc. There are two types of stents that are presently utilized: permanent stents and temporary stents. A permanent stent is designed to be maintained in a body lumen for an indeterminate amount of time. Temporary stents are designed to be maintained in a body lumen for a limited period of time in order to maintain the patency of the body lumen, for example, after trauma to a lumen caused by a surgical procedure or an injury. Permanent stents are typically designed to provide long-term support for damaged or traumatized wall tissues of the lumen.

It is known that permanent stents, over time, can become encapsulated and covered with endothelium tissues, for example, in cardiovascular applications. Similarly, permanent stents are known to become covered by epithelium, for example, in urethral or other applications. Temporary stents, on the other hand, are designed to maintain the passageway of a lumen open for a specific, limited period of time, and preferably do not become incorporated into the walls of the lumen by tissue ingrowth or encapsulation. Temporary stents can advantageously be eliminated from body lumens after a predetermined, clinically appropriate period of time, for example, after the traumatized tissues of the lumen have healed and a stent is no longer needed to maintain the patency of the lumen. For example, temporary stents can be used as substitutes for in-dwelling catheters for applications in the treatment of prostatic obstruction or other urethral stricture diseases. Another indication for temporary stents in a body lumen is after energy ablation, such as laser or thermal ablation, or irradiation of prostatic tissue, in order to control post-operative acute urinary retention or other body fluid retention. As another example, a temporary stent can be used to treat endoluminal leaks or perforations, such as in the esophagus, for example, in an effort to protect an endoluminal injury from enteric contents of a body during the healing process. The stent can be removed when the injury has healed and meanwhile the patient can continue with oral intake, for example.

It is known in the art to make both permanent and temporary stents from various conventional, biocompatible materials, such as metals. However, there are several disadvantages that can be associated with the use of conventional stents. For example, it is known that the metal stents can become encrusted, encapsulated, epithelialized or ingrown with body tissue. The stents are known to migrate on occasion from their initial insertion location. Such stents are known to cause irritation to the surrounding tissues in a lumen, especially during insertion or removal. Regardless of whether the stent is categorized as permanent or temporary, if the stent has been encapsulated, epithelialized, etc., the surgical removal of the stent can result in undesirable pain and discomfort to the patient and possibly additional trauma to the lumen tissue. In addition to the pain and discomfort, the patient can be subjected to an additional time consuming and complicated surgical procedure with the attendant risks of surgery, in order to remove the stent. Similar complications and problems, as in the case of metal stents, can well result when using stents made from non-absorbable biocompatible polymer or polymer-composites, although these materials can offer certain benefits such as reduction in stiffness. It is known to use bioabsorbable and biodegradable materials for manufacturing temporary stents. The conventional bioabsorbable or bioresorbable materials from which such stents can be made may be selected to absorb or degrade over time, which can eliminate the need for subsequent surgical procedures to remove the stent from the body lumen. However, there are disadvantages and limitations known to be associated with the use of bioabsorbable or biodegradable stents. The limitations can arise from the characteristics of the stent or the materials from which such stents are made. One of the problems that can be associated with current stents is that the materials break down too quickly. This improper breakdown or degradation of a stent into large, rigid fragments in the interior of a lumen, such as the urethra or esophagus, can cause obstruction to normal flow, such as voiding, thereby interfering with the primary purpose of the stent in providing lumen patency. Alternatively, they can take a long time to breakdown and can stay in the target lumen for a considerable period of time after their therapeutic use has been accomplished. As another example, esophageal stents, biodegradable or otherwise, can migrate within or even distally from the esophagus as a result of the unique muscle movements in that area of the body, or other factors, which can lead to emergency surgery in some circumstances. Also, there are no known stents with wires approved by the FDA for esophageal use. Accordingly, there is a need in this art for novel, temporary stents, wherein the stents remain functional in a body lumen for the duration of a prescribed, clinically appropriate period of time to accomplish the appropriate therapeutical purpose, and can then be removed with minimal discomfort for the patient and without the need for a surgical procedure.

### BRIEF SUMMARY OF THE INVENTION

The invention disclosed and taught herein is directed to improved apparatuses, systems and methods for temporary intraluminal stents.

A stent can comprise a flexible elongated tubular body having proximal and distal ends, a wall with an outer surface, and a seam formed in the wall of the body between the proximal and distal ends. In at least one embodiment, the seam can be unseamed in vivo so that upon removal the cross-sectional area is reduced to facilitate extraction from the lumen. Advantageously, the seam can be formed in a helix to facilitate a reduced cross-sectional area upon removal. The stent can include one or more flexible peripheral wires coupled to the body, which can be memory or shape-retaining wires, and can include one or more longitudinal support wires. The stent can be a removable intraluminal stent, can have one or more cross-sectional areas along its length and can include one or more layers.

The disclosure also provides a method of inserting a temporary intraluminal stent into a lumen of a body using a sheath that can comprise providing a tubular stent including a radially compressible body having two ends with a helical seam there between, the stent defining a cross-sectional area when in a radially uncompressed state, decreasing the cross-sectional area of the stent, inserting at least a portion of the stent into the sheath, inserting at least a portion of the stent into the lumen, removing the stent from the sheath, and allowing the stent to return to the cross-sectional area so that at least a portion of the stent contacts the lumen.

The disclosure also provides a method of removing a stent from a lumen, the stent having an elongated tubular body having a cross-sectional area and two ends with a helical seam there between and the seam having an unseaming member disposed adjacent one end of the body, the method comprising grasping the unseaming member, applying force to the unseaming member thereby unseaming at least a portion of the seam, and removing the stent from the lumen.

The disclosure also provides a method of removing a stent from a lumen, the stent having an elongated tubular body having two ends with a helical seam there between, the method comprising unseaming at least a portion of the seam, allowing at least a portion of the body to form a helix, grasping the stent, and removing the stent from the lumen.

The disclosure also provides a removable intraluminal stent that can comprise an elongated tubular body having an outer surface, proximal and distal ends, and a longitudinal axis, at least one wire coupled peripherally about the body and a seam disposed between the proximal and distal ends of the body.

The disclosure also provides a method of forming an intraluminal stent that can comprise providing an obliquely-angled-parallelogram-shaped sheet of body material having a proximal edge, a distal edge and two longitudinal edges, the proximal and distal edges being obliquely angled to the longitudinal edges, and coupling the longitudinal edges to one another so that the sheet forms a tubular body having an outer surface.

### BRIEF DESCRIPTION OF THE SEVERAL VIEWS OF THE DRAWINGS

FIG. 1 is a perspective schematic view of one of many embodiments of a removable intraluminal stent utilizing certain aspects of the present invention.

FIG. 1A is a schematic view of one of many types of wire having bending points that can, but need not, be used in one or more embodiments of the present invention.

FIG. 2 is a side schematic view of the stent of FIG. 1.

FIG. 3 is an end schematic view of the stent of FIG. 2.

FIG. 4 is a side schematic view of the stent of FIG. 2 in a sheet form.

FIG. 5 is a side schematic view of the stent of FIG. 2 partially assembled.

FIG. 6 is a cross-sectional schematic view of a lumen with the stent coupled to an insertion tool being inserted into the lumen.

FIG. 7 is a cross-sectional schematic view of a lumen with the stent coupled to the lumen.

FIG. 8 is a cross-sectional schematic view of a lumen with another exemplary embodiment of the stent coupled to the lumen.

FIG. 9 is a cross-sectional schematic view of a lumen with the stent being uncoupled from the lumen.

FIG. 10 is a cross-sectional schematic view of the lumen with the stent of FIG. 9 being removed from the lumen.

FIG. 11 is a side schematic view of another of many embodiments of the stent having a non-uniform cross-sectional area.

FIG. 12 is a side schematic view of another of many embodiments of the stent having a non-uniform cross-sectional area with a reduced central region.

FIG. 13 is a side schematic view of another of many embodiments of the stent having a linear seam.

### DETAILED DESCRIPTION

The Figures described above and the written description of specific structures and functions below are not presented to limit the scope of what Applicant has invented or the scope of the appended claims. Rather, the Figures and written description are provided to teach any person skilled in the art to make and use the invention for which patent protection is sought. Those skilled in the art will appreciate that not all features of a commercial embodiment of the invention is described or shown for the sake of clarity and understanding. Persons of skill in this art will also appreciate that the development of an actual commercial embodiment incorporating aspects of the present invention will require numerous implementation-specific decisions to achieve the developer's ultimate goal for the commercial embodiment. Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would be, nevertheless, a routine undertaking for those of skill in this art having benefit of this disclosure. It must be understood that the invention disclosed and taught herein is susceptible to numerous and various modifications and alternative forms. Lastly, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the Figures and are not intended to limit the scope of the invention or the appended claims. The term “couple,” “coupled,” “coupling,” “coupler,” and like terms are used broadly herein and can include any method or device for securing, binding, bonding, fastening, attaching, joining, inserting therein, forming thereon or therein, communicating, or otherwise associating, for example, mechanically, magnetically, electrically, chemically, directly or indirectly with intermediate elements, one or more pieces of members together and can further include without limitation integrally forming one functional member with another in a unity fashion. The coupling can occur in any direction, including rotationally. The terms “endoscope,” “endoscopic” and like terms are used broadly in this application and include any tool insertable into a body having a channel through which tools and other devices can be placed or used, whether inserted through a natural body orifice or through an artificially created opening, such as through an incision or other procedure, and thus includes a laparoscope or other instruments and apparatuses usable for purposes expressly or impliedly discussed herein. The term “wire(s),” “wire mesh” and like terms are used broadly in this application and include wire formed from any material including, but not limited to, metal, alloy or plastic, and that can have any suitable cross-section including, without limitation, round, elliptical, square, rectangular and other geometric shapes, and that can be solid or hollow.

Applicant has created a removable intraluminal stent for treating injuries or other conditions inside a body lumen. The stent can include a seam, such as a helical seam, for supporting the structure of the stent, which can be unseamed, for example, during removal of the stent from a lumen. The stent can include an elongated tubular member, or stent body, for positioning inside a lumen. The stent can be flexible and can include one or more other stent components coupled to the body. The stent can include a peripheral wire for coupling the stent in place and/or a longitudinal support wire for supporting one or more components of the stent. The present invention will now be described in more detail with reference to the Figures.

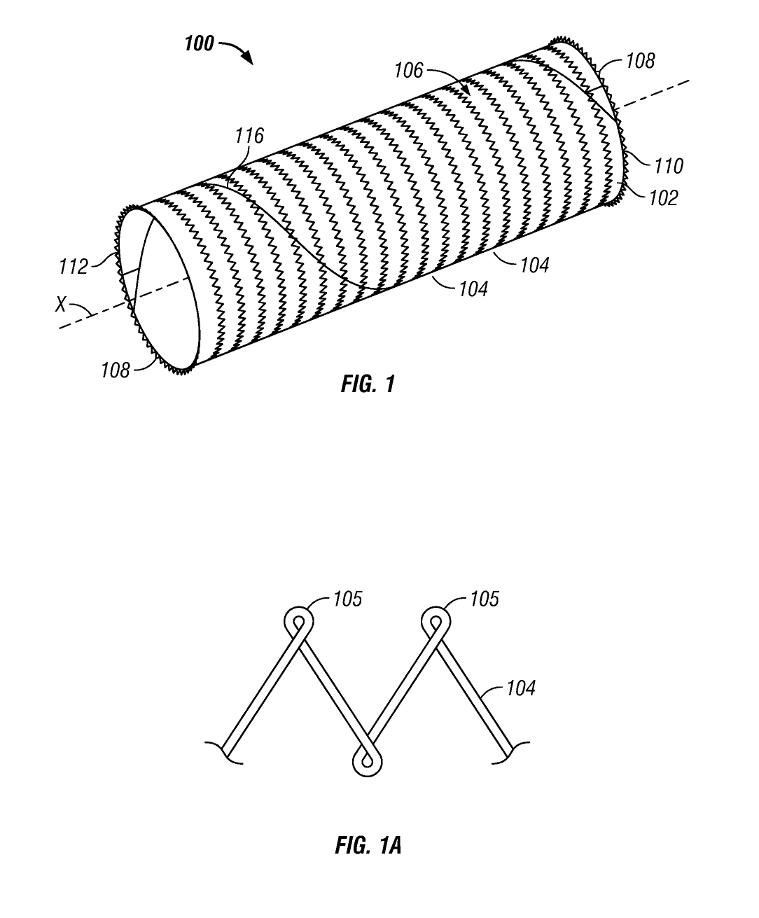

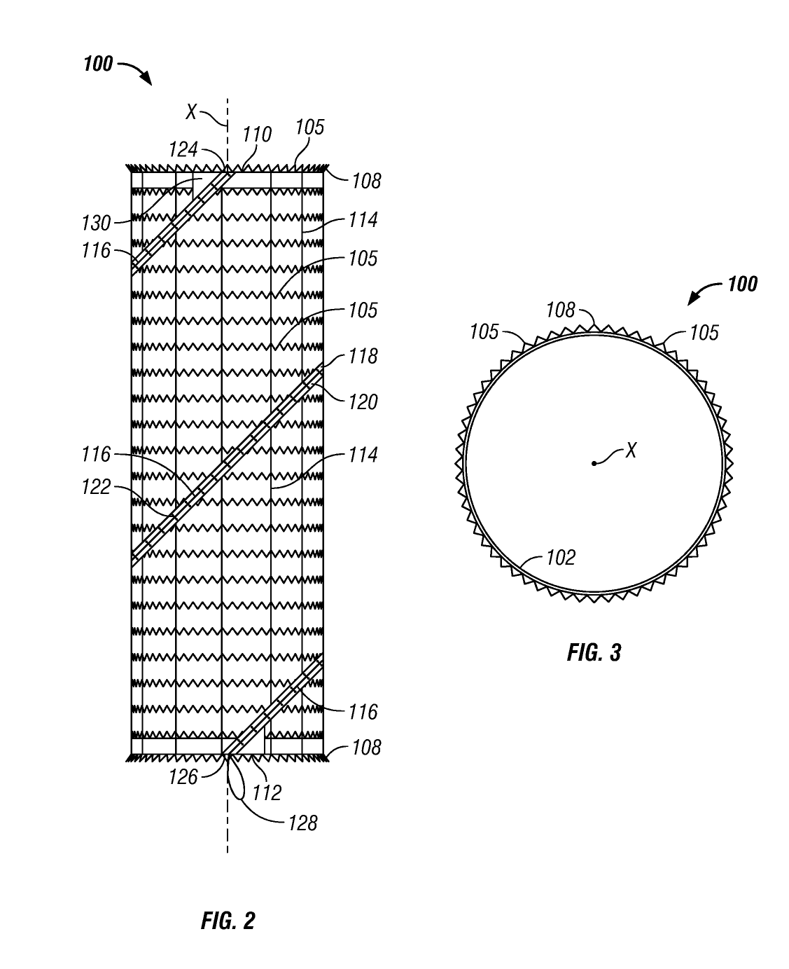

FIG. 1 is a perspective schematic view of one of many embodiments of a removable intraluminal stent utilizing certain aspects of the present invention. FIG. 1A is a schematic view of one of many types of wire having bending points that can, but need not, be used in one or more embodiments of the present invention. FIG. 2 is a side schematic view of the stent of FIG. 1. FIG. 3 is an end schematic view of the stent of FIG. 2. FIGS. 1-3 will be described in conjunction with one another. Stent 100 can include an elongated tubular member, such as body 102, for positioning inside a lumen to be treated. Body 102 can flex, contract and expand, and stent 100 can include one or more other components coupled to body 102. Body 102 can be formed from any material, such as a nondegradeable or nonpermeable material and, in at least one embodiment, can preferably be formed from silicone or other like material, in whole or in part. Body 102 can include one or more layers, as will be further described below, and can be of any length, cross-sectional area or shape required by a particular application. In at least one exemplary embodiment, which is but one of many, stent 100 can, but need not, have one or more outside cross-sectional dimensions equal to or larger than an inside cross-sectional dimension of a lumen into which stent 100 can be coupled. While the embodiment of stent 100 shown in FIGS. 1-3, which is but one of many, is shown to be cylindrical, it need not be and can be any cross-sectional shape. The cross-sectional shape, dimensions or area of stent 100 can, but need not, change along its length and can be defined by body 102 singularly or in combination with other components, as further described below.

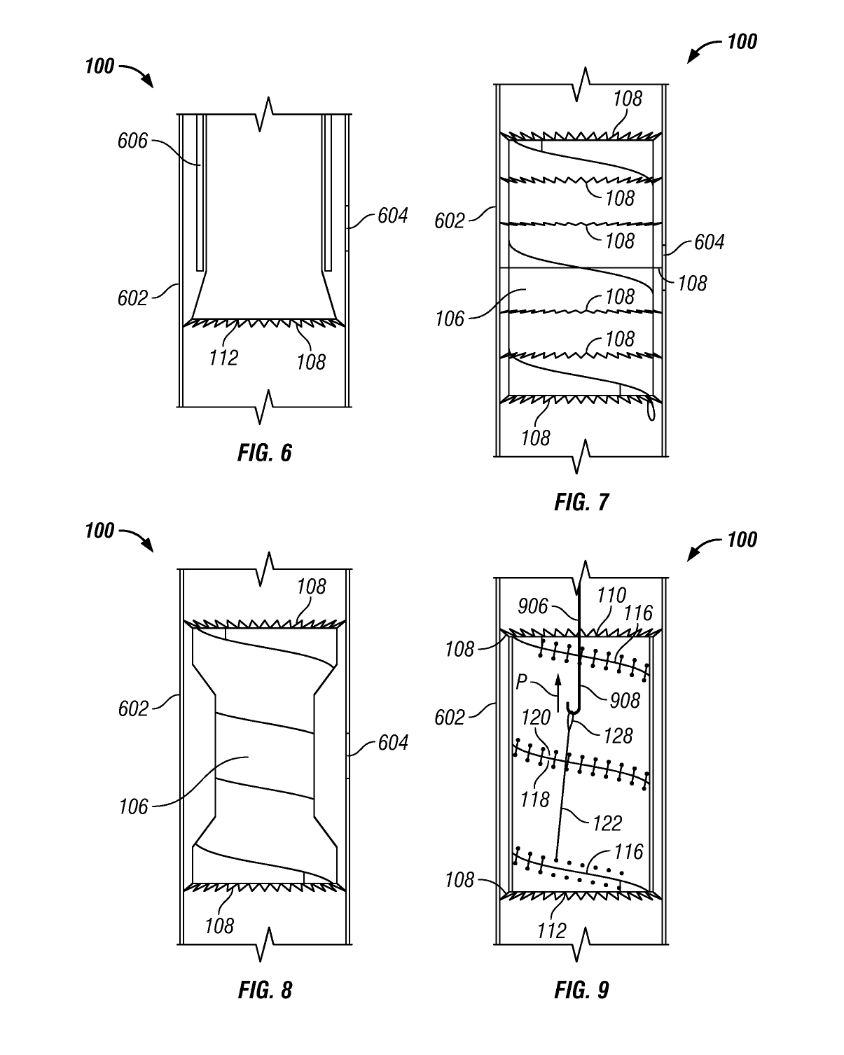

Stent 100 can include one or more wires coupled to body 102. One or more peripheral wires 104 can be coupled to body 102, for example, along its length, such as for coupling the stent in place, supporting the structure of stent 100 or for allowing stent 100 to contract or expand. Wires 104 can be coupled to body 102 to form one or more rings about the periphery of body 102, such as, for example, to form a series of rings concentric about central longitudinal Axis X of stent 100. Peripheral wires 104 can be coupled to the radial exterior of body 102, such as to body outer surface 106, which can, but need not, be the radially outermost surface of stent 100. Alternatively, wires 104 can be formed integrally with body 102, such as being embedded in the wall of body 102. As another example, body 102 can be formed from two or more layers, such as inner and outer shells, and one or more wires 104 can be coupled therebetween. One or more peripheral wires 104, such as, but not limited to, the endmost wires 104, can be retaining wires 108, such as for retarding or preventing migration of stent 100 within a lumen. Retaining wire(s) 108, or portions thereof, can protrude radially outwardly from the outermost surface of stent 100, such as for contacting the inside surface of the lumen (see, e.g., FIGS. 6-9). Retaining wire(s) 108 can extend away from Axis X and can be at any angle relative to Axis X required by a particular application, including perpendicular, and can for example form an angle of about 45 degrees with Axis X. While stent 100 is shown to include two retaining wires 108 in the embodiment of FIGS. 1-3, one at proximal end 110 and one at distal end 112, stent 100 can include any number of retaining wires 108, including a single retaining wire 108, and retaining wire(s) 108 can be coupled at any location along the length of stent 100, as required by a particular application. Wires 104, including one or more retaining wires 108, can be formed from any material required by a particular application and can preferably, but need not, be formed from a memory shape-retaining material, such as a memory shape-retaining metal alloy. Wires 104 can be of any suitable shape or cross-section, as described above, and can advantageously be sinusoidal, or substantially sinusoidal. Wires 104 can, but need not, include bending points 105, such as pre-weakened points, flexible portions, crests or, as another example, loops (see FIG. 1A), for bending or creating a spring-like structure or response. For example, each wire 104 can be coupled to body 102, so that stent 100 can deform in response to a force or forces in the radially inward direction and then return or expand to its normal shape or cross-sectional area once the force(s) cease.

With reference to FIG. 2, stent 100 can, but need not, include one or more support wires 114 for supporting one or more components of stent 100 or for providing overall structure, support, or integrity to stent 100, singularly or in combination with other components. For example, support wire(s) 114 can, but need not, be coupled to body 102, so that they are parallel, or substantially parallel, to central Axis X when stent 100 is in an assembled form. Similarly to peripheral wires 104, each support wire 114 can be coupled to body 102 integrally, to outer surface 106, or otherwise. Each support wire 114 can be coupled to one or more peripheral wires 104 (including retaining wires 108), for example, for positioning each peripheral wire 104 along the length of stent 100 or for keeping peripheral wires 108 from gathering or bunching together. Each wire, 104, 108, 114 can be coupled in any manner required by a particular application, such as, for example, by welding, brazing, gluing, bending, twisting or another manner, to one another or to another component of stent 100. Support wires 114 can, but need not, be perpendicular to peripheral wires 104, and can be formed from the same or a different material than wires 104, which can be any material required by a particular application. Alternatively, some embodiments of stent 100 can include a wire mesh (not shown), such as a readily available wire mesh, in lieu of the combination of wires 104, 108, 114 in whole or in part, as will be understood by one of ordinary still in the art. One of ordinary skill will understand that the wire mesh can be equivalent to the combination of wires 104, 108, 114 for purposes of the present invention and that each aspect of the combination of wires 104, 108, 114 discussed herein can apply to a wire mesh. The choice of material, weight or gauge of each wire 104, 108, 114 or wire mesh will be a matter of design for a particular application and can vary between embodiments, applications, or within a single embodiment based on any number of factors, such as the lumen to be treated or, as another example, the material of body 102. In any event, it is an object of the present invention that at least some embodiments of stent 100 remain able to flex, contract and expand, wherein the stiffness, deformability and other physical characteristics of stent 100 can vary as required by a particular application.

Referring to FIGS. 1 and 2, stent 100 can include a seam 116 for assembling stent 100, for example, by coupling or uncoupling two edges of stent 100, in whole or in part. For example, longitudinal edges 118 and 120 can be coupled to form seam 116, removably, permanently or otherwise. Edges 118, 120 can, but need not, be reinforced for purposes of forming seam 116, for example, by strengthening each edge 118, 120. Seam 116 can be any type of seam required by a particular application and can include coupling in any manner, such as by tying, zipping, gluing, sticking or, as another example, stitching. Seam 116 can, but need not, be helical, as will be further discussed below. For example, seam 116 can be parallel to Axis A, in whole or in part, or can be any shape required by a particular application. Seam 116 can support stent 100, for example, during installation of stent 100 and while stent 100 is coupled to a lumen. Seam 116 can be unseamed, in whole or in part, such as during removal of stent 100. For example, unseaming can allow stent 100 to at least partially deform, which can reduce its cross-sectional area from an assembled form to facilitate removal from the lumen, as will be further described below. In at least one of many embodiments, such as the embodiment of FIGS. 1-3, stent 100 can include a wire or string, such as a suture 122, for coupling edges 118, 120 to form seam 116. Suture 122 can be formed from any material required by a particular application, such as a synthetic nonabsorbable polypropylene, for example, Prolene® or another suitable material. Suture 122 can form seam 116, such as by coupling edges 116, 118, for example, by stitching, and can include a fixedly coupled end 124 and a removably coupled end 126. For example, ends 124, 126 can, but need not, be coupled to proximal and distal ends 110, 112, respectively, of stent 100. Removably coupled end 126 can include an unseaming member 128, such as a loop, tab, or other member that can be adapted to initiate unseaming of seam 116, singularly or in combination with other components. Stent 100 can include one or more reinforcement tabs 130 for reinforcing one or more portions of stent 100, such as body 102. Tab 130 can be formed from any material required by a particular application, such as silicone or plastic, and can include a protective coating. Tab 130 can be adapted for coupling one or more components of stent 100 thereto, such as, for example, ends 124, 126 of suture 122, and/or can resist wear or breakage of a portion of stent 100, such as a corner or edge. In at least one advantageous embodiment, for example, tab 130 can dull or blunt a corner of stent 100 for making removal of stent 100 less traumatic.

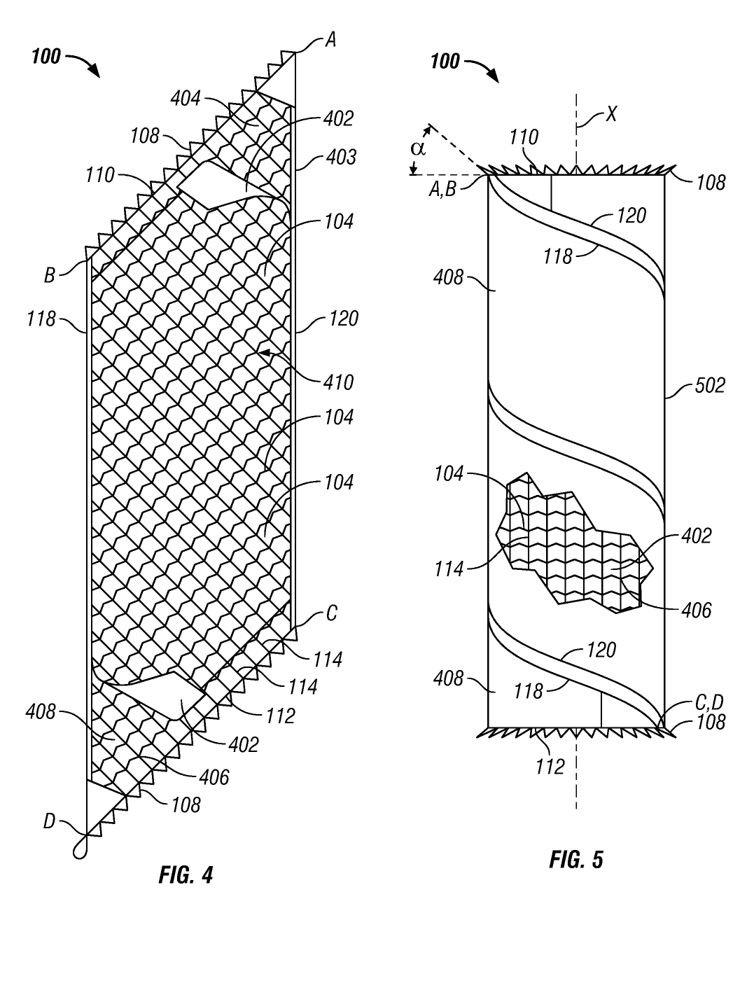

FIG. 4 is a side schematic view of the stent of FIG. 2 in a sheet form. As described above, stent 100 can, but need not, include one or more layers, such as an inner layer 402 and an outer layer 404 of body 102. Inner layer 402 is shown to be transparent and peeled back at the upper and lower corners of FIG. 4 for illustrative purposes only and can be any configuration or color required by a particular application. Inner layer 402 can be formed from a sheet 403 of any material required by a particular application, for example, silicone, plastic, polypropylene, resin or another material, separately or in combination. Outer layer 404 and/or inner layer 402 can, but need not, include two or more sub-layers, such as a wire layer 406 or an outer shell 408. Outer shell 408 can be formed from the same or a different material as inner layer 402, such as silicone or plastic, and can, but need not, be the radially outermost layer of stent 100. Wire layer 406 can include wire mesh 410, one or more peripheral wires 104, or one or more structural wires 114, separately or in combination, as described above. Alternatively, wire layer 406 can be formed integrally with material sheet 403, for example, so that the inner and outer layers 402, 404 form one integral layer that forms the wall of body 102. Stent 100 can include one or more peripheral wires 104 that can be retaining wires 108, which are shown in FIG. 4 to be coupled to stent 100 at the proximal end 110 and distal end 112, but which can be coupled at any position and in any number along the length of stent 100. For example, one or more retaining wires 108 can, but need not, be coupled to the sheet form of stent 100 parallel to the proximal or distal edges, which will form the proximal and distal ends of assembled stent 100, so that upon assembly, the one or more wires 108 are concentric about Axis X. Retaining wires 108 can preferably be coupled so that at least a portion of each wire 108 extends radially outwardly from the outermost surface of stent 100 about Axis X, for example, so that the one or more retaining wires 108 define an outermost retaining dimension for contacting the interior surface of a lumen. Stent 100 can include any number of layers or sub-layers required by a particular application, each of which can comprise any number of materials, in whole in part, and the coupling of which can form body 102 of stent 100.

With further reference to FIG. 4, the sheet form of stent 100 can be any shape required by a particular application. In at least one advantageous embodiment, such as the embodiment of FIG. 4, which is but one of many, stent 100 can preferably be obliquely-angled-parallelogram-shaped in sheet form, but can be any shape, for example, rhomboidal, parallelogram-shaped, rectangular, or another shape. As shown in FIG. 4, stent 100 can have four corners, labeled A, B, C and D for illustrative purposes, each formed by adjacent edges, wherein each corner can form any angle required by a particular application. For example, adjacent edges can form angles between about 15 degrees and about 75 degrees, and can advantageously form angles between about 30 degrees and about 60 degrees, and more advantageously form angles between about 40 degrees and about 50 degrees. The sheet form of stent 100 can be manipulated to form an assembled form of stent 100, for example, by coupling longitudinal edges 118, 120, in whole or in part. The relationship between corners A and B (and similarly between corners C and D), in the assembled form can determine the intraluminal shape of stent 100. For example, corners A and B (and similarly C and D if applicable) can be coupled at a single point, such as a point in the same plane, so that an elongated tubular body can be formed having parallel planar ends (see, e.g., FIG. 5). However, this need not be the case and each corner A-D can be coupled in any relation relative to one another to form any shape of stent 100 required by a particular application.

FIG. 5 is a side schematic view of the stent of FIG. 2 partially assembled. With reference to FIGS. 4 and 5, a particular sheet form of stent 100 (e.g., FIG. 4) can be adapted to form one or more other forms of stent 100, such as a partially assembled form (e.g., FIG. 5), a fully assembled form (e.g., FIGS. 7, 8) or another form. For example, longitudinal edges 118 and 120 can be brought together or adjacent one another (see FIG. 4), such as by bringing corners A and B and corners C and D, respectively, together about Axis X so that an elongated tubular body 502 (FIG. 5) can be formed concentric about Axis X. In an embodiment such as the one shown in FIG. 5, which is but one of many, edges 118, 120 can abut one another in a helical fashion longitudinally to form a cylindrical tubular stent 100 having a helically-shaped seam. Edges 118, 120 can be coupled to form a seam in any fashion required by a particular application thereby holding stent 100 in an assembled form, temporarily, permanently or otherwise. Wires 104, 108, if present, can be reoriented relative to Axis X (compared to the angles shown in FIG. 4), for example, when corners A and B, and C and D, are brought together to form assembled stent 100. As shown in FIG. 5, for example, inner layer 402 can be the radially innermost layer of stent 100, outer shell 408 can be the radially outermost layer of stent 100, and wire layer 406 can be coupled there between. Alternatively, however, stent 100 can include a body formed from one layer, for example, having integrally formed wires and inner and outer surfaces, or any number of layers, or types of layers, required by a particular application. Each peripheral wire 104 and support wire 114 can, but need not, be coupled between inner layer 402 and outer shell 408, for example, so that the radially outermost surface of stent 100 comprises only the material from which outer shell 408 is formed. However, one or more peripheral wires 104, such as retaining wires 108, may not be covered by outer shell 408 and can preferably protrude radially outwardly from the outermost surface of stent 100 thereby defining the outermost dimension (or largest cross-sectional area) of stent 100. For example, as shown in FIG. 5, retaining wires 108 can, but need not, be coupled to stent 100 on the proximal and distal ends 110, 112 of stent 100, so that retaining wires 108 protrude radially outwardly from the outermost surface of stent 100 at an angle α relative to the horizontal, which can be any angle, and can advantageously be 45 degrees. It should be noted that stent 100 can be any shape or size, in any form, required by a particular application, and that the embodiments shown in FIGS. 4-5 are but two of many and are described herein only for illustrative purposes, as will be understood by one of ordinary skill in the art. For example, while body lumens can typically be cylindrical, some may not be and, in any event, a cross-section of stent 100 can be shaped or adapted as required by a particular application.

FIG. 6 is a cross-sectional schematic view of a lumen with the stent coupled to an insertion tool being inserted into the lumen. Lumen 602 can have a defect 604, such as a hole, injury, or other condition, and stent 100 can be coupled to lumen 602 during treatment of the condition. Stent 100 can be coupled to lumen 602 using an insertion tool, for example, sheath 606, such as a sheath having a smaller cross-sectional area than stent 100 and lumen 602, or any other coupler tool required by a particular application, such as an endoscopic coupler tool. As an illustration, a particular embodiment of stent 100 can have an outermost retaining dimension that can, but need not, be equal to or greater than the inside dimension of sheath 606. The cross-sectional area of at least a portion of stent 100 can be reduced, such as by radially compressive force(s) including, for example, folding, so that stent 100 can be coupled at least partially within sheath 606, as illustrated in FIG. 6. Stent 100 and sheath 606 can be inserted, for example, into lumen 602, such as an esophagus or other lumen, so that the distal end 112 of stent 100 is located distally from defect 604, such as an esophageal hole, or other area of treatment. Stent 100 can be uncoupled from sheath 606, such as being pushed or pulled therefrom. As stent 100 is uncoupled from sheath 606, for example, one or more portions of stent 100, such as retaining wire 108, can expand radially, such as to contact the inner surface of lumen 602 for holding stent 100 in place. Sheath 606 can be any sheath required by a particular application and any tool, such as an endoscopic tool, can be used to uncouple stent 100 from sheath 606, as will be understood by one of ordinary skill in the art. Stent 100 can have any number of retaining wires 108 coupled along its length for contacting the inner wall of lumen 602 and, in at least one embodiment, can have at least one retaining wire 108 at each end. For example, one or more distal retaining wires 108 can be coupled to lumen 602 distally from defect 604 and one or more proximal retaining wires 108 can be coupled to lumen 602 proximally from defect 604 thereby coupling stent 100 within lumen 602. Each retaining wire 108 can have any radial dimension required by a particular application and can preferably have a radial dimension equal to or slightly greater than the inside radial dimension of lumen 602, for example, so that stent 100 can be securely coupled within lumen 602.

FIG. 7 is a cross-sectional schematic view of a lumen with the stent coupled to the lumen. FIG. 8 is a cross-sectional schematic view of a lumen with another exemplary embodiment of the stent coupled to the lumen. FIGS. 7 and 8 will be described in conjunction with one another. Stent 100 can have any number of peripheral wires 104, 108 required by a particular application, separately or in combination, or can have none at all. If present, the number of wires 104, 108 can depend on any number of factors, such as, for example, the type of lumen 602 or defect 604, the location of the defect 604, or other factors, separately or in combination. For example, the embodiment shown in FIG. 7, which is but one of many, can have four retaining wires 108 for coupling stent 100 to lumen 602. As another example, two retaining wires 108 can be coupled proximally to defect 604 and two retaining wires 108 can be coupled distally from defect 604. Generally, the greater the number of retaining wires 108, the more securely stent 100 can be coupled to lumen 602. An embodiment such as shown in FIG. 7, for example, can protect defect 604 from both the proximal and distal directions. As an example, where stent 100 is coupled to a lumen such as an esophagus, stent 100 can protect defect 604 from substances flowing distally, such as ingested food, as well as from substances flowing proximally, such as enteric contents or reflux. Outer surface 106 can, but need not, contact the inner surface of lumen 602, in whole or in part, separately or in combination with each retaining wire 108.

With reference to FIGS. 7 and 8, stent 100 can, but need not, have the same cross-sectional area or shape along the entire length of body 102. For example, stent 100 can have a uniform cross-sectional area along its length, such as the embodiment shown in FIG. 7, or stent 100 can have a changing cross-sectional area along its length, for example, the “hour-glass” shape shown in FIG. 8, or in FIG. 12, described below. As other examples, stent 100 can have polygonal cross-sections or can be shaped like a truncated cone, wherein no two radial cross-sections have the same area, shown for example in FIG. 11, described below. The embodiment shown in FIG. 8 includes two retaining wires 108 and a recessed mid-section and can be used, for example, where contact between stent 100 and lumen 602 is desirous both proximally and distally from defect 604, but where contact with lumen 602 can be less desirous between the retaining wires 108. For example, defect 604 can have swelling or tissue adjacent thereto, with which contact with stent 100 can be undesirable, or other reasons can exist for using an embodiment such as FIG. 8 for a particular application, as will be understood by one of ordinary skill in the art. As another example, at least one embodiment of stent 100 can include only one retaining wire 108, or no retaining wire.

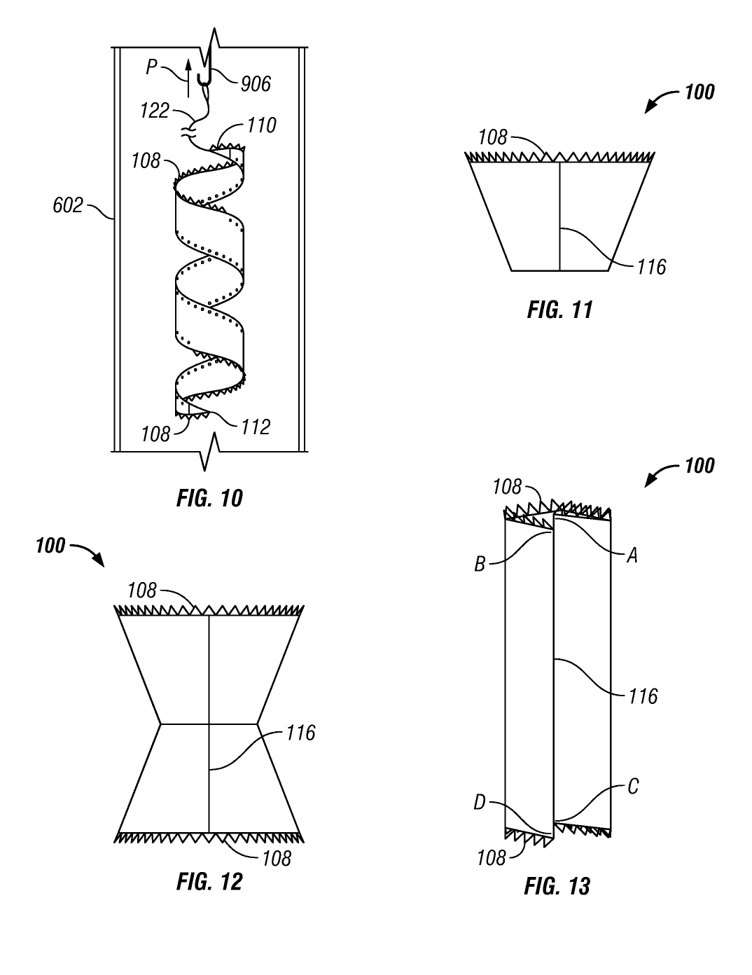

FIG. 9 is a cross-sectional schematic view of a lumen with the stent being uncoupled from the lumen. Stent 100 can include seam 116, for example, between its proximal end 110 and distal end 112 for aiding in the removal of stent 100 from lumen 602. Seam 116 can, but need not, be helical and, alternatively, can be any type of seam required by a particular application. For example, seam 116 can include a zipper, stitches, or other couplers, separately or in combination. In at least one embodiment, such as the embodiment illustrated in FIGS. 9 and 10 for illustrative purposes, stent 100 can preferably include a suture 122 for sewing or stitching together edges 118, 120 to form seam 116 and the in vivo form of stent 100. Suture 122 can include an unseaming member 128, such as, for example, a loop, knot or other structure for initiating unseaming upon removal of stent 100. A removal tool 906, such as an endoscopic tool, can be inserted at least partially into lumen 602 so that tool 906 can be coupled to stent 100. Tool 906 can have a coupler end 908 adapted to couple to stent 100, which can include any coupling structure required by a particular application, such as, for example, a hook, clamp, grabber, grasper, or other structure. Tool 906 and coupler end 908 can be adapted to couple to any portion of stent 100, for example, body 102, seam 116 or another portion, such as suture 122. As shown in the exemplary embodiments of FIGS. 9 and 10 for illustrative purposes, coupler end 908 can be coupled to unseaming member 128, such as, for example, at the distal end 112 of stent 100, and unseaming member 128 can be moved in an unseaming direction using tool 906. The unseaming direction is shown in FIGS. 9 and 10 to be the proximal direction, as indicated by Arrow P, but can be any direction. As unseaming member 128 is moved in the unseaming direction, seam 116 can unseam, as illustrated at the distal end 112 of stent 100 in FIG. 9. As unseaming member 128 moves in the unseaming direction, seam 116 can unseam, gradually or otherwise, which can allow adjacent edges 118, 120 to at least partially uncouple.

FIG. 10 is a cross-sectional schematic view of the lumen with the stent of FIG. 9 being removed from the lumen. Stent 100 can begin to at least partially change to a sheet form (e.g., FIGS. 9 and 10), for example, by unraveling in a helical fashion, which can reduce one or more cross-sectional areas of stent 100. The cross-sectional area of stent 100 can be reduced to an area less than the inside cross-sectional area of lumen 602, which can allow stent 100 to uncouple from lumen 602, such as by at least partially reducing contact between stent 100 and lumen 602. For example, one or more retaining wires 108 can uncouple from lumen 602. Additionally, or alternatively, rotational torque can be applied to stent 100, which can reduce the cross-sectional area of at least a portion of stent 100. Suture 122 can, but need not, be anchored to stent 100, for example, at proximal end 110, and can, but need not, be used to fully remove stent 100 from lumen 602. Alternatively, any tool can be coupled to any portion of stent 100 to remove stent 100 from lumen 602. Because the cross-sectional area of stent 100 can be reduced as described above, there can be reduced contact between portions of stent 100 and lumen 602 during removal. For example, retaining wires 108 may not drag along the inner surface of lumen 602, which can result in less irritation, pain, or discomfort for the patient being treated.

At least one other exemplary embodiment of stent 100 will now be described. FIG. 11 is a side schematic view of another of many embodiments of the stent having a non-uniform cross-sectional area. FIG. 12 is a side schematic view of another of many embodiments of the stent having a non-uniform cross-sectional area with a reduced central region. FIGS. 11 and 12 will be described in conjunction with one another. In at least one exemplary embodiment, such as the one shown in FIG. 11, stent 100 can, but need not, include a retaining wire 108. For example, stent 100 can be shaped like a truncated cone, or another shape, and can have a linear or substantially linear is seam 116. Alternatively, seam 116 can have any form required by a particular application, for example, helical, zig-zag, or another form. Referring to FIG. 12, stent 100 can, but need not, have two retaining wires 108, each of which can, but need not, define the same cross-sectional area, as required by a particular application. Stent 100, such as the embodiment of FIG. 12, can be formed from one or more sheets of material. For example, the embodiment of FIG. 12 can be formed from a single sheet of material or, as another example, can be formed from two sheets of material folded into cones, or other shapes, and coupled together, removably or otherwise, to form stent 100.

FIG. 13 is a side schematic view of another of many embodiments of the stent having a linear seam. Stent 100 can include one or more helically coupled retaining wires 108. For example, a sheet form of stent 100 can include two proximal corners A and B (and similarly two distal corners C and D) that are not coupled at the same point in a particular assembled form of stent 100, as compared to the embodiment shown in FIG. 5. Seam 116 can, but need not, be linear and can be any length required by a particular application. For example, the length of seam 116 in a particular embodiment of stent 100 can, but need not, be determined by the angles formed by adjacent edges at corners A-D of stent 100 in a particular sheet form, as will be understood by one of ordinary skill in the art. In practice, the length of stent 100 would generally be sufficient to cover defect 604 or another condition.

Other and further embodiments utilizing one or more aspects of the invention described above can be devised without departing from the spirit of Applicant's invention. For example, adjacent edges of the stent can be at any angle, including perpendicular, and the stent can be any length. Moreover, the stent can have any cross-sectional shape and the seam need not be helical. Further, the various methods and embodiments of the intraluminal stent can be included in combination with each other to produce variations of the disclosed methods and embodiments. Discussion of singular elements can include plural elements and vice-versa.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The invention has been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by Applicant, but rather, in conformity with the patent laws, Applicant intends to fully protect all such modifications and improvements that come within the scope or range of equivalent of the following claims.

What is claimed is:

1. A stent, comprising:
a flexible elongated tubular body having proximal and distal ends and a wall with an outer surface; and
a helical seam formed in the wall of the body between the proximal and distal ends.

2. The stent of claim 1, wherein the tubular body is formed from an obliquely-angled-parallelogram-shaped sheet comprising two longitudinal edges coupled to form the helical seam.

3. The stent of claim 1, further comprising a plurality of flexible peripheral wires coupled along the length of the body.

4. The stent of claim 3, wherein at least one of the wires is a shape-retaining wire.

5. The stent of claim 4, wherein the shape-retaining wire is made from a memory shape-retaining metal alloy.

6. The stent of claim 3, wherein at least one of the wires is a substantially sinusoidal wire.

7. The stent of claim 3, wherein at least one of the wires is embedded in the wall of the tubular member.

8. The stent of claim 3, further comprising a plurality of longitudinally disposed support wires coupled to each peripheral wire.

9. The stent of claim 3, wherein the stent is an intraluminal stent and wherein at least one of the wires is adapted to contact a lumen to at least minimize migration of the stent within the lumen.

10. The stent of claim 1, wherein the seam includes a suture having two opposite ends.

11. The stent of claim 10, wherein the suture is formed from synthetic nonabsorbable polypropylene.

12. The stent of claim 10, wherein one end of the suture is fixedly coupled to the tubular body and the opposite end of the suture is removably coupled to the tubular body, the removably coupled end having a loop.

13. The stent of claim 12, wherein the end of the suture having the loop is adapted to originate unseaming of the seam.

14. The stent of claim 1, wherein the tubular body is plastic.

15. The stent of claim 1, further comprising a series of wires disposed peripherally along the length of the body and an outer shell having an inner surface coupled to the body radially outwardly from the body outer surface so that at least some of the wires are disposed between the body outer surface and the shell inner surface.

16. The stent of claim 1, wherein the cross-sectional area of the tubular member changes along its length.

17. A method of inserting a temporary intraluminal stent into a lumen of a body using a sheath, comprising:
providing a tubular stent including a radially compressible body having two ends with a helical seam there between, the stent defining a cross-sectional area when in a radially uncompressed state;
decreasing the cross-sectional area of the stent;
inserting at least a portion of the stent into the sheath;
inserting at least a portion of the stent into the lumen;
removing the stent from the sheath; and
allowing the stent to return to the cross-sectional area so that at least a portion of the stent contacts the lumen.

18. The method of claim 17, wherein providing a tubular stent further comprises providing a plurality of peripheral wires coupled along the length of the body.

19. The method of claim 17, further comprising pushing the stent from the sheath.

20. A method of removing a stent from a lumen, the stent having an elongated tubular body having a cross-sectional area and two ends with a helical seam there between, the seam having an unseaming member disposed adjacent one end of the body, the method comprising:
grasping the unseaming member;
applying force to the unseaming member thereby unseaming at least a portion of the seam; and
removing the stent from the lumen.

21. The method of claim 20, further comprising reducing the cross-sectional area of the stent.

22. The method of claim 20, further comprising applying rotational torque to the stent to reduce the cross-sectional area of the stent.

23. A method of removing a stent from a lumen, the stent having an elongated tubular body having two ends with a helical seam there between, the method comprising:
unseaming at least a portion of the seam;
allowing at least a portion of the body to form a helix;
grasping the stent; and
removing the stent from the lumen.

24. The method of claim 23, further comprising decreasing a cross-sectional area of the helix by applying rotational torque to the stent.

25. The method of claim 23, wherein grasping the stent includes inserting at least a portion of a grasping tool into the lumen.

26. A removable intraluminal stent, comprising:
an elongated tubular body having an outer surface, proximal and distal ends, and a longitudinal axis;
at least one wire coupled peripherally about the body; and
a seam disposed between the proximal and distal ends of the body.

27. The stent of claim 26, wherein the seam is disposed helically about the body between the proximal and distal ends.

28. The stent of claim 26, further comprising a wire mesh coupled to the body, the wire mesh comprising a series of wires disposed peripherally about the body and at least one support wire disposed about the body substantially parallel to the body longitudinal axis.

29. A method of forming an intraluminal stent, comprising:
providing an obliquely-angled-parallelogram-shaped sheet of body material having a proximal edge, a distal edge and two longitudinal edges, the proximal and distal edges being obliquely angled to the longitudinal edges; and
coupling the longitudinal edges to one another so that the sheet forms a tubular body having an outer surface.

30. The method of claim 29, wherein the sheet of body material is rhomboidal.

31. The method of claim 29, wherein coupling the longitudinal edges to one another further comprises forming a helical seam.

32. The method of claim 29, further comprising coupling at least one flexible peripheral wire to the sheet so that the wire is substantially parallel to the sheet proximal and distal edges.

33. The method of claim 32, wherein the at least one peripheral wire is a substantially sinusoidally-shaped memory wire.

34. The method of claim 32, further comprising coupling at least one support wire to the sheet so that the support wire is substantially perpendicular to the at least one peripheral wire.

35. The method of claim 29, further comprising coupling a peripheral wire to the sheet adjacent to at least one of the proximal and distal edges so that at least a portion of the wire protrudes radially outwardly from the outer surface of the tubular body.

36. The method of claim 29, further comprising reinforcing the longitudinal edges of the sheet and coupling the longitudinal edges to one another with a suture having two ends thereby forming a seam.

37. The method of claim 36, further comprising fixedly coupling one end of the suture adjacent one edge of the sheet and forming a loop in the other end of the suture, wherein the end of the suture having the loop is adapted to initiate unseaming of the seam.

38. The method of claim 29, further comprising coupling at least one reinforcement tab to the sheet.

39. The method of claim 29, further comprising forming the sheet of body material from a material selected from the group consisting of silicone, plastic, polymers, polypropylenes and resins.
